(19) United States
(12) Patent Application Publication
(10) Pub. No.: US 2005/0153416 A1
(43) Pub. Date: Jul. 14, 2005

(54) Conjugates of the c domain of human gelatinase a and polyethylene glycol, methods of purification and uses thereof

(76) Inventors: Nikolaos Dimoudis, Wielenbach (DE); Erhard Kopetzki, Penzberg (DE); Martin Lanzendoerfer, Muenchen (DE); Werner Scheuer, Penzberg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 10/505,990

(22) PCT Filed: Feb. 28, 2003
(86) PCT No.: PCT/EP03/02099

(30) Foreign Application Priority Data
Mar. 2, 2002 (EP) 02004799.9

(51) Int. Cl. C12N 9/64

(52) U.S. Cl. 435/226

(57) ABSTRACT

There is provided a conjugate comprising the C domain of human gelatinase A (PEX domain, PEX) and one to three poly(ethylene glycol) group(s), said poly(ethylene glycol) group(s) having an overall weight of from about 10 to 40 kDa and being conjugated via primary amino group(s) of said PEX. The conjugates are useful as anticancer agents.

This invention relates to conjugates of the C domain of human gelatinase A (PEX domain, PEX) with polyethylene glycol (PEG), pharmaceutical compositions thereof, methods for the production and purification and methods for use.

### BACKGROUND OF THE INVENTION

The matrix metalloproteinases (MMPs) constitute a family of proteolytic enzymes that together can degrade all components of the extracellular matrix and basement membranes. MMP activity plays a major role during physiological and pathological processes including embryogenesis, metastasis and inflammatory diseases (Nicolson, G. L., et al., Curr. Opin. Cell Biol. 1 (1989) 1009-10019; Stetler-Stevenson, W. G., et al., Annu. Rev. Cell Biol. 9 (1993) 541-573; Docherty, A. J., and Murphy, G., Ann. Rheum. Dis. 49 (1990) 469-479; and Woessner, J. F., Jr., FASEB J. 5 (1991) 2145-2154). MMPs are organized in distinct structural domains including an NH₂-terminal zymogen domain, a catalytic domain and a C-terminal domain which is also called a hemopexin-like domain.

Gelatinase A (72-kDa gelatinase, MMP-2, EC3.4.24.24) is a member of the family of said matrix metalloproteinases. Gelatinase A is described in the Swiss-Prot Database under No. P08253 and by Collier, I. E., et al., Genomics 9 (1991) 429-434. Its inactive form is called progelatinase A which contains a prodomain blocking its active site.

Cell-surface activation of progelatinase A occurs in a complex between progelatinase A, TIMP-2 and MT1-MMP. MT1-MMP acts on MMP2 by cleaving its prodomain and thus activating this enzyme (cf. Murphy, G., et al., APMIS 107 (1999) 33-44; Nagase, H., and Woessner, J. F., J. Biol. Chem. 274 (1999) 21491-21494). Extracellular matrix degradation by activated MMP2 is one of the prerequisites for the escape of tumor cells from a solid tumor and the formation of metastases (Cockett, M. I., et al., Biochem. Soc. Symp. 63 (1998) 295-313).

Based on this mechanism it is assumed that the inhibition of gelatinase A might prevent growth of primary tumors, tumor cell invasion and metastasis as well as it might inhibit angiogenesis (Pfeifer, A., et al., Proc. Natl. Acad. Sci. USA 97 (2000) 12227-12232; Silletti, S., et al., Proc. Natl. Acad. Sci. USA 98 (2001) 119-124). Such an inhibitor could also be an isolated PEX domain as suggested by Brooks, P. C., et al., Cell 92 (1998) 391-400.

PEX binds to integrin avb3 and inhibits the binding of intact MMP2 (Brooks, P., et al., Cell 85 (1996) 683-693). Furthermore, PEX inhibits angiogenesis and tumor growth (Brooks, P., et al., Cell 92 (1998) 391-400). Pfeifer, A., et al., in Proc. Natl. Acad. Sci. USA 97 (2000) 12227-12232, describe the suppression of angiogenesis by lentiviral delivery of PEX. Bello, L., et al., in Cancer Res. 61 (2001) 7501-7506, describe chemotherapy combined with PEX and the inhibition of glioma angiogenesis, cell proliferation and invasion by PEX. However, PEX treatment did not produce an improvement in survival and induced severe side-effects.

Covalent modification of proteins with poly(ethylene glycol) (PEG) has proven to be a method for extending the circulating half-lives of some proteins in the body (Hershfield, M. S., et al., New England Journal of Medicine 316 (1987) 589-596; and Meyers, F. J., et al., Clin. Pharmacol. Ther. 49 (1991) 307-313). Other advantages of PEGylation are an increase of solubility and a decrease in protein immunogenicity (Katre, N. V., J. Immunol. 144 (1990) 209-213). A method for the PEGylation of proteins is the use of poly(ethylene glycol) activated with amino-reactive reagents like N-hydroxysuccinimide (NHS). With such reagents poly(ethylene glycol) is attached to the proteins at free primary amino groups such as the N-terminal α-amino group and the ε-amino groups of lysine residues. However, a major limitation of this approach is that proteins typically contain a considerable amount of lysine residues and therefore the polyethylene groups are attached to the protein in a non-specific manner at all of the free ε-amino groups, resulting in a heterologous product mixture of random PEGylated proteins. Therefore, many NHS-PEGylated are unsuitable for commercial use because of low specific activity and heterogeneity and it is not possible to predict what influence the PEGylation of a certain polypeptide will have. Inactivation results from covalent modification of one or more lysine residues or the N-terminal amino residue required for biological activity or from covalent attachment of the poly(ethylene glycol) residues near or at the active site of the protein. For example, it was found that modification of human growth hormone using NHS-PEGylation reagents reduces the biological activity of the protein by more than 10-fold (Clark, R., et al., J. Biol. Chem. 271 (1996) 21969-21977). Human growth hormone contains 9 lysines in addition to the N-terminal amino acid. Certain of these lysines are located in regions of the protein known to be critical for receptor binding (Cunningham, B. C., et al., Science 254 (1991) 821-825). In addition, the modification of erythropoietin by the use of amino-reactive poly(ethylene glycol) reagents results also in a nearly complete loss of biological activity (Wojchowski, D. M., et al., Biochim. Biophys. Acta 910 (1987) 224-232). Covalent modification of Interferon-α2 with amino-reactive PEGylation reagents results in 40-75% loss of bioactivity (U.S. Pat. No. 5,382,657). A similar modification of G-CSF results in greater than 60% loss of activity (Tanaka, H., et al., Cancer Research 51 (1991) 3710-3714) and of Interleukin-2 in greater than 90% loss of bioactivity (Goodson, R. J., and Katre, N. V., BioTechnology 8 (1990) 343-346).

It was an object of the present invention to find a form of the PEX protein which has an acceptable therapeutic activity and efficacy for tumor treatment and which is capable of increasing the survival of a patient suffering from cancer.

### SUMMARY OF THE INVENTION

The present invention provides conjugates consisting of PEX being covalently linked to from one to three polyethylene glycol (PEG) groups (PEGylated PEX) by PEGylation of primary amino groups of PEX. Such conjugates surprisingly have a positive influence on the survival rate in tumor treatment compared to the treatment with unmodified PEX.

The present invention therefore provides a conjugate comprising the C domain of human gelatinase A (PEX domain, PEX) and one to three poly(ethylene glycol) group(s), said poly(ethylene glycol) group(s) having an overall molecular weight of from about 10 to 40 kDa and being conjugated via primary amino group(s) of said PEX.

According to the invention, poly(ethylene glycol) group(s) preferably has/have the formula
—CO—(CH₂)ₓ—(OCH₂CH₂)ₘOR
and said —CO group forms an amide bond with one of the amino groups of said PEX, wherein
x is 2 or 3;
m is from about 220 to about 900 dependent on the molecular weight of the poly(ethylene glycol) group(s);
R is lower alkyl.

According to the invention, preferred conjugates have the formula I
P—[NHCO—(CH₂)ₓ—(OCH₂CH₂)ₘ—OR]ₙ (I)
wherein
x, R, and m are defined as above, n is 1 to 3, and
P is said PEX without the n amino group(s) which form amide linkage(s) with the poly(ethylene glycol) group(s).

Also preferred are conjugates which comprise branched PEG.

The invention further comprises methods for the production of the conjugates according to the invention.

The invention further comprises pharmaceutical compositions containing a conjugate according to the invention.

The invention further comprises methods for the production of pharmaceutical compositions containing a conjugate according to the invention in a pharmaceutically effective amount.

The invention further comprises the use of a conjugate according to the invention for the preparation of a medicament useful in the treatment of cancer.

The invention further comprises methods for the treatment of human cancer (e.g. breast, lung, prostate or colon cancer) characterized in that a pharmaceutically effective amount of amino-reactive PEGylated PEX is administered in one to seven bolus applications per week to the patient in need thereof.

### DETAILED DESCRIPTION OF THE INVENTION

Amino-reactive PEGylation as used herein designates a method of randomly attaching poly(ethylene glycol) chains to primary amino group(s) of the target protein PEX by the use of reactive poly(ethylene glycol), preferably by the use of N-hydroxysuccinimidyl esters of, preferably, methoxypoly(ethylene glycol). The coupling reaction preferentially attaches poly(ethylene glycol) to reactive primary amino groups like the ε-amino groups of lysine residues or the α-amino group of the N-terminal amino acid of PEX. Such amino group conjugation of PEG to proteins is widely known in the state of the art. A review of such methods is given by, for example, Veronese, F. M., Biomaterials 22 (2001) 405-417. According to Veronese, the conjugation of PEG to primary amino groups of proteins can be performed by using activated PEGs which perform an alkylation of said primary amino groups. For such a reaction, activated alkylating PEGs, for example PEG aldehyde, PEG-tresyl chloride or PEG epoxide can be used. Further useful reagents are acylating PEGs such as hydroxysuccinimidyl esters of carboxylated PEGs or PEGs in which the terminal hydroxy group is activated by chloroformates or carbonylimidazole. Further useful PEG reagents are PEGs with amino acid arms. Such reagents can contain the so-called branched PEGs, whereby at least two identical or different PEG molecules are linked together by a peptidic spacer (preferably lysine) and, for example, bound to PEX as activated carboxylate of the lysine spacer.

Another method for amino group PEGylation consists in that in a first step the protein is covalently reacted with a bifunctional reagent which contains an amino-reactive group which binds to the primary amino groups of the protein and contains a preferably protected thiol group. After binding of the reagent to the protein, the thiol group is reacted with a thiol-reactive form of poly(ethylene glycol). Such activated PEGs are also known in the art and are, for example, PEG-orthopyridyl-disulfide, PEG-maleimide, PEG-vinylsulfone, and PEG-iodoacetamide.

“Amino-reactive PEGylated PEX”, “amino-reactive PEGylation” or “PEGylated PEX” as used herein therefore means that PEX has attached covalently one, two or three poly(ethylene glycol) groups by amino-reactive coupling to the PEX molecule. The groups can be attached at different sites of the PEX molecule which are primary amino groups, preferably at the most reactive sites, e.g., the ε-amino groups of the lysine side chains or the N-terminal α-amino group. Due to the synthesis method used, PEGylated PEX can consist of a mixture of mono-, di- and/or triPEGylated PEX, whereby the sites of PEGylation can be different in different molecules or can be substantially homogeneous in regard to the amount of poly(ethylene glycol) side chains per molecule and/or the site of PEGylation in the molecule.

In the following and in the examples, some of these reagents which are preferred for the production of amino-reactive PEGylated PEX are described. It is understood that modifications, for example, based on the methods described by Veronese, F. M., Biomaterials 22 (2001) 405-417, can be made in the procedures as long as coupling occurs between PEG and PEX via the primary amino groups of PEX, and one to three PEGs with an overall molecular weight of about 10 to 40 kDa are coupled.

The occurrence of several potentially reactive primary amino groups in the target protein (for PEX there are lysines +1 terminal amino acid) leads to a series of PEGylated PEX isomers that differ in the point of attachment of the poly(ethylene glycol) chain and will be called positional isomers in the following. The attachment site in a single PEX molecule is not clearly predicted and for that reason referred to as random.

The invention provides amino-reactive PEGylated forms of PEX with improved properties. Such PEGylated PEX contains one to three PEG groups linear or branched randomly attached thereto, whereby the overall molecular weight of all PEG groups in the conjugate is 10 to 40 kDa. By “molecular weight” as used here there is to be understood the mean molecular weight of the PEG; the term “about” indicates that in said PEG preparations, some molecules will weigh more and some less than the stated molecular weight.

This implies that the PEGylated forms of PEX according to the invention comprise, for example,
monoPEGylated PEX, the PEG group having a molecular weight of about 20 to 40 kDa;
diPEGylated PEX, the PEG groups having a molecular weight of about 10 kDa to 20 kDa each;
triPEGylated PEX, the PEG groups having a molecular weight of about 10 kDa each,
   or mixtures thereof.

“PEG or PEG group” according to the invention means a residue containing poly(ethylene glycol) as an essential part. Such a PEG can contain further chemical groups which are necessary for binding reactions; which results from the chemical synthesis of the molecule; or which is a spacer for optimal distance of the parts of the molecule from one another. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462. Especially preferred are PEGs with two PEG side-chains (PEG2) linked via the primary amino groups of a lysine (Monfardini, C., et al., Bioconjugate Chem. 6 (1995) 62-69).

“Substantially homogeneous” as used herein means that the only PEGylated PEX molecules produced, contained or used are those having one, two or three PEG group(s) attached and are homogeneous in regard to the site of PEGylation. The preparation may contain unreacted (i.e., lacking PEG group) protein. As ascertained by peptide mapping and N-terminal sequencing, one example below provides for the preparation which is at least 90% PEG-PEX conjugate (preferably monoPEGylated) and at most 5% unreacted protein. Isolation and purification of such homogeneous preparations of PEGylated PEX can be performed by usual purification methods, preferably size exclusion chromatography.

“MonoPEGylated” as used herein means that PEX is PEGylated at only one amino group per PEX molecule, whereby only one PEG group is attached covalently at this site and the sites of attachment can vary within the monoPEGylated species. The monoPEGylated PEX is at least 90% of the preparation, and most preferably, the monoPEGylated PEX is 92%, or more, of the preparation. The monoPEGylated PEX preparations according to the invention are therefore homogeneous enough to display the advantages of a homogeneous preparation, e.g., in a pharmaceutical application.

PEGylation of PEX can be performed according to the methods of the state of the art.

In a preferred embodiment of the invention, the conjugates of the invention may be represented by formula (I)
P—[NHCO—(CH₂)ₓ—(OCH₂CH₂)ₘ—OR]ₙ (I)
wherein in this formula and in the following, P is an PEX protein as described herein, (i.e. without the amino group or amino groups which form an amide linkage with the carbonyl shown in formula (I); and wherein in this formula and in the following, R is lower alkyl; x is 2 or 3; n is from 1 to 3; and n and m are chosen so that the molecular weight of the conjugate minus the PEX protein is from 10 kDa to 40 kDa.

As used herein and in the following, “lower alkyl” means a linear or branched alkyl group having from one to six carbon atoms. Examples of lower alkyl groups include methyl, ethyl and isopropyl. In accordance with this invention, R is any lower alkyl. Conjugates in which R is methyl are preferred.

The symbol “m” represents the number of ethylene oxide groups (OCH₂CH₂) in the poly(ethylene oxide) group. A single PEG subunit of ethylene oxide has a molecular weight of about 44 daltons. Thus, the molecular weight of the conjugate (excluding the molecular weight of the PEX) depends on the number “m”. As used herein, the given ranges of “m” merely have an orientational meaning. The ranges of “m” are determined in any case, and exactly, by the molecular weight of the PEG group(s). In the conjugates of this invention “m” is from about 220 to about 900 (corresponding to a molecular weight of about 10 kDa to about 40 kDa). The number m is selected within this range such that the resulting conjugate of this invention has a physiological activity comparable to unmodified PEX, which activity may represent the same as, more than, or a fraction of the corresponding activity of unmodified PEX. A molecular weight of “about” a certain number means that it is within a reasonable range of that number as determined by conventional analytical techniques. The number “m” is selected so that the molecular weight of each poly(ethylene glycol) group covalently linked to the PEX protein is from about 10 kDa to about 40 kDa, however the maximum molecular weight of all poly(ethylene glycol) groups together not exceeding 40 kDa.

In the conjugates of this invention, the number “n” is the number of poly(ethylene glycol) groups covalently bound to free amino groups (including ε-amino groups of a lysine amino acid and/or the amino-terminal amino group) of an PEX protein via amide linkage(s). A conjugate of this invention may have one, two, or three PEG groups per molecule of PEX. “n” is an integer ranging from 1 to 3, preferably “n” is 1 or 2, and more preferably “n” is 1. If the PEG molecule is a branched PEG, n is understood as the number of attached branched PEG molecules. If, for example, a branched PEG contains two PEG molecules, n=2 denotes that two branched PEGs (consequently, four PEG molecules) are attached to PEX, however not exceeding the given molecular weight range.

Compounds of formula (I) can be prepared, for example, from a known activated polymeric material: 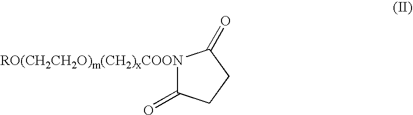
in which R and m are as described above, by condensing the compound of formula II with the PEX protein. Preferred compounds of formula (II) in which x is 3 are alpha-lower alkoxybutyric acid succinimidyl esters of poly(ethylene glycol) (lower alkoxy-PEG-SBA). Compounds of formula (II) in which x is 2 are alpha-lower alkoxypropionic acid succinimidyl esters of poly(ethylene glycol) (lower alkoxy-PEG-SPA). Any conventional method of reacting an activated ester with an amine to form an amide can be utilized. In the reaction described above, the exemplified succinimidyl ester is a leaving group causing the amide formation. The use of succinimidyl esters such as the compounds of formula II to produce conjugates with proteins are disclosed in U.S. Pat. No. 5,672,662.

When the PEGylation reagent was combined with a succinimidyl ester compound of Formula II, it has been found that at a pH of about 7.0, a protein:PEG ratio of about 1:3, and a reaction temperature of from 20-25° C., a mixture of mono-, di-, and trace amounts of the tri-PEGylated species were produced. When the protein:PEG ratio was about 1:1, primarily the mono-PEGylated species is produced. By manipulating the reaction conditions (e.g., ratio of reagents, pH, temperature, protein concentration, time of reaction etc.), the relative amounts of the different PEGylated species can be varied.

MonoPEGylated PEX can also be produced according to the methods described in WO 94/01451. WO 94/01451 describes a method for preparing a recombinant polypeptide with a modified terminal amino acid alpha-carbon reactive group. The steps of the method involve forming the recombinant polypeptide and protecting it with one or more biologically added protecting groups at the N-terminal alpha-amine and C-terminal alpha-carboxyl. The polypeptide can then be reacted with chemical protecting agents to selectively protect reactive side chain groups and thereby prevent side chain groups from being modified. The polypeptide is then cleaved with a cleavage reagent specific for the biological protecting group to form an unprotected terminal amino acid alpha-carbon reactive group. The unprotected terminal amino acid alpha-carbon reactive group is modified with a chemical modifying agent. The side chain protected terminally modified single copy polypeptide is then deprotected at the side chain groups to form a terminally modified recombinant single copy polypeptide. The number and sequence of steps in the method can be varied to achieve selective modification.

Further preferred conjugates according to the invention consist of PEX protein being covalently linked to from one to three lower-alkoxy poly(ethylene glycol) groups, each poly(ethylene glycol) group being covalently linked to the protein via a linker of the formula —C(O)—X—S—Y— with the C(O) of the linker forming an amide bond with one of said amino groups, X is —(CH₂)ₖ— or —CH₂(O—CH₂—CH₂)ₖ—, k is from 1 to 10, Y is 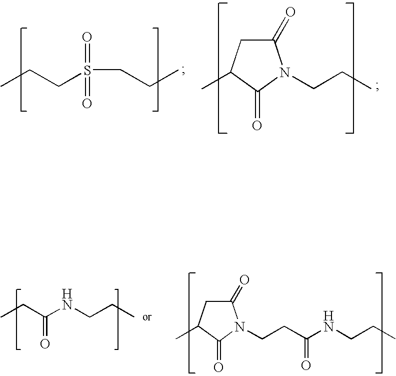
the average molecular weight of each poly(ethylene glycol) moiety is from about 20 kDa to about 40 kDa, not exceeding 40 kDa for all poly(ethylene glycol) moieties, and the molecular weight of the conjugate is from about 44 kDa to about 64 kDa at a molecular weight of 24 kDa for PEX polypeptide, or from about 48 kDa to about 68 kDa at a molecular weight of 28 kDa for PEX glycoprotein.

These PEX conjugates may also be represented by formula (III)
P—[NH—CO—X—S—Y—(OCH₂CH₂)ₘ—OR]ₙ (III)
wherein R may be any lower alkyl, by which is meant a linear or branched alkyl group having from one to six carbon atoms such as methyl, ethyl, isopropyl, etc. A preferred alkyl is methyl. X may be —(CH₂)ₖ— or —CH₂(O—CH₂—CH₂)ₖ—, wherein k is from 1 to about 10. Preferably, k is from 1 to about 4, more preferably, k is 1 or 2. Most preferably, X is —(CH₂).

In formula III, Y is

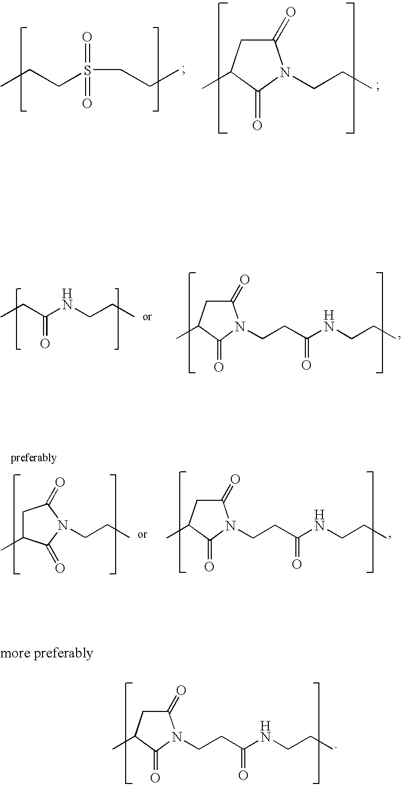

In formula (III), the number m is selected such that the resulting conjugate of formula (III) has a physiological activity comparable to unmodified PEX, which activity may represent the same as, more than, or a fraction of the corresponding activity of unmodified PEX. m represents the number of ethylene oxide chains in the PEG unit. A single PEG subunit of —(OCH₂CH₂)— has a molecular weight of about 44 daltons. Thus, the molecular weight of the conjugate (excluding the molecular weight of the PEX) depends on the number m. A molecular weight of “about” a certain number means that it is within a reasonable range of that number as determined by conventional analytical techniques. m is therefore an integer ranging from about 220 to about 900 (corresponding to a molecular weight of from about 10 to 40 kDa).

In formula (III), the number n is the number of ε-amino groups of a lysine amino acid in a PEX protein covalently bound to a PEG unit via an amide linkage. A conjugate of this invention may have one, two, or three PEG units per molecule of PEX. n is an integer ranging from 1 to 3, preferably n is 1 or 2, and more preferably n is 1.

Preferred PEX proteins of formula (III) are represented by the formulae:

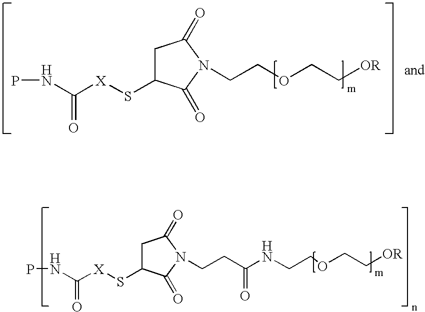

Most preferred PEX protein products are represented by the formula:

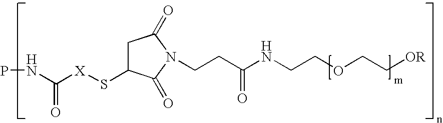

Such PEX conjugates may be prepared by
(a) covalently reacting a primary amino group of an PEX protein represented by the formula, P—[NH₂]ₙ, with a bifunctional reagent represented by the formula
   Z-CO—X—S-Q (amino-reactive coupling)
to form an intermediate with an amide linkage represented by the formula:
   P—[NH—CO—X—S-Q]ₙ
wherein P is an PEX protein less the amino group which forms an amide linkage; n is an integer ranging from 1 to 3; Z is a reactive group, e.g. a carboxylic-NHS ester; X is —(CH₂)ₖ— or —CH₂(O—CH₂—CH₂)ₖ—, wherein k is from 1 to about 10; and Q is a protecting group, like alkanoyl, e.g. acetyl;
(b) covalently reacting the intermediate with an amide linkage from step (a) with an activated poly(ethylene glycol) derivative represented by the formula
   W—[OCH₂CH₂]ₘ—OR
to form an PEX protein product represented by the formula: 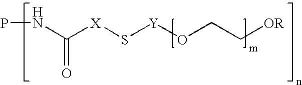
wherein W is a sulfhydryl reactive form of Y; m is an integer ranging from about 220 to about 900 (corresponding to a molecular weight of the PEG residue of 10 to 40 kDa); R is lower alkyl; and Y is as defined above.

In this embodiment, the bifunctional reagent is preferably N-succinimidyl-S-acetylthiopropionate or N-succinimidyl-S-acetylthioacetate, Z is preferably N-hydroxy-succinimide, and the activated poly(ethylene glycol) derivative W-[OCH₂CH₂]ₘ—OR is preferably selected from the group consisting of iodo-acetyl-methoxy-PEG, methoxy-PEG-vinylsulfone, and methoxy-PEG-maleimide.

In general, the PEX conjugates of formula (III) may be prepared by amino-reactive covalent linking of thiol groups to PEX (“activation”) and coupling the resulting activated PEX with a poly(ethylene glycol) (PEG) derivative. The first step comprises covalent linking of thiol groups via NH₂-groups of PEX. This activation of PEX is performed with bifunctional reagents which carry a protected thiol group and an additional reactive group, such as active esters (e.g., a succinimidylester), anhydrides, esters of sulphonic acids, halogenides of carboxylic acids and sulphonic acids, respectively. The thiol group is protected by groups known in the art, e.g., acetyl groups. These bifunctional reagents are able to react with the ε-amino groups of the lysine amino acids by forming an amide linkage.

In a preferred embodiment the activation of the ε-amino lysine groups is performed by reaction with bifunctional reagents having a succinimidyl moiety. The bifunctional reagents may carry different spacer species, e.g. —(CH₂)ₖ— or —CH₂—(O—CH₂—CH₂—)ₖ— moieties, wherein k is from 1 to about 10, preferably from 1 to about 4, and more preferably 1 or 2, and most preferably 1.

Examples of these reagents are N-succinimidyl-S-acetylthiopropionate (SATP) and N-succinimidyl-S-acetylthioacetate (SATA) 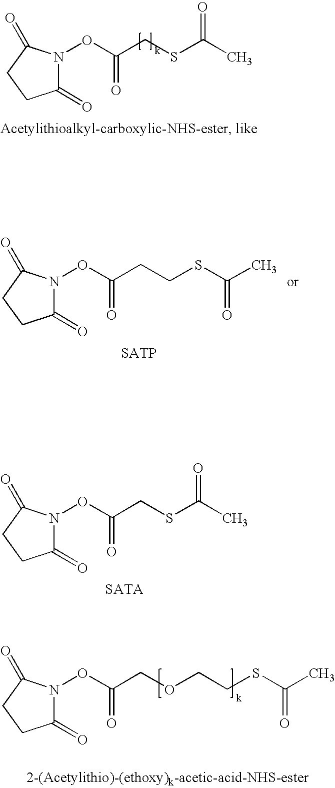
with k as defined above.

The preparation of the bifunctional reagents is known in the art. Precursors of 2-(acetylthio)-(ethoxy)ₖ-acetic-acid-NHS-esters are described in DE 39 24 705, while the derivatization to the acetylthio compound is described by March, J., Advanced Organic Chemistry (1977) 375-376. SATA is commercially available (Molecular Probes, Eugene, Oreg., USA and Pierce, Rockford, Ill.).

The number of thiol groups to be added to an PEX molecule can be selected by adjusting the reaction parameters, i.e., the protein (PEX) concentration and the protein/bifunctional reagent ratio. Preferably, the PEX is activated by covalently linking from 1 to 5 thiol groups per PEX molecule, more preferably from 1.5 to 3 thiol groups per PEX molecule. These ranges refer to the statistical distribution of the thiol group over the PEX protein population.

The reaction is carried out, for example, in an aqueous buffer solution, pH 6.5-8.0, e.g., in 10 mM potassium phosphate, 300 mM NaCl, pH 7.3. The bifunctional reagent may be added in DMSO. After completion of the reaction, preferably after 30 minutes, the reaction is stopped by addition of lysine. Excess bifunctional reagent may be separated by methods known in the art, e.g., by dialysis or column filtration. The average number of thiol groups added to PEX can be determined by photometric methods described in, for example, Grasetti, D. R,. and Murray, J. F. in J. Appl. Biochem. Biotechnol. 119 (1967) 41-49.

The above reaction is followed by covalent coupling of an activated poly(ethylene glycol) (PEG) derivative. Suitable PEG derivatives are activated PEG molecules with an average molecular weight of from about 10 to about 40 kDa.

Activated PEG derivatives are known in the art and are described in, for example, Morpurgo, M., et al. J. Bioconj. Chem. 7 (1996) 363 ff for PEG-vinylsulfone. Linear chain and branched chain PEG species are suitable for the preparation of the compounds of Formula 1. Examples of reactive PEG reagents are iodo-acetyl-methoxy-PEG and methoxy-PEG-vinylsulfone:

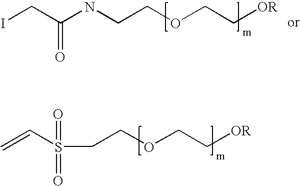

The use of these iodo-activated substances is known in the art and described e.g. by Hermanson, G. T., in Bioconjugate Techniques, Academic Press, San Diego (1996) p. 147-148.

Most preferably, the PEG species are activated by maleimide using (alkoxy-PEG-maleimide), such as methoxy-PEG-maleimide (MW 10000 to 40000; Shearwater Polymers, Inc.). The structure of alkoxy-PEG-maleimide is as follows: 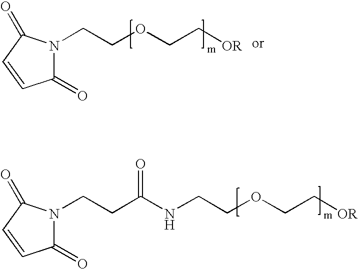
with R and m are as defined above, preferably

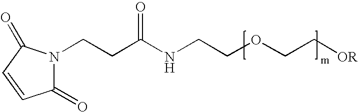

The coupling reaction with alkoxy-PEG-maleimide takes place after in situ cleavage of the thiol protecting group in an aqueous buffer solution, e.g. 10 mM potassium phosphate, 300 mM NaCl, 2 mM EDTA, pH 6.2. The cleavage of the protecting group may be performed, for example, with hydroxylamine in DMSO at 25° C., pH 6.2 for about 90 minutes. For the PEG modification the molar ratio of activated PEX/alkoxy-PEG-maleimide should be from about 1:1 to about 1:6. The reaction may be stopped by addition of cysteine and reaction of the remaining thiol (—SH) groups with N-methylmaleimide or other appropriate compounds capable of forming disulfide bonds. Because of the reaction of any remaining active thiol groups with a protecting group such as N-methylmaleimide or other suitable protecting group, the PEX proteins in the conjugates of this invention may contain such protecting groups. Generally the procedure described herein will produce a mixture of molecules having varying numbers of thiols protected by different numbers of the protecting group, depending on the number of activated thiol groups on the protein that were not conjugated to PEG-maleimide.

Whereas N-methylmaleimide forms the same type of covalent bond when used to block the remaining thiol-groups on the PEGylated protein, disulfide compounds will lead in an intermolecular sulfide/disulfide exchange reaction to a disulfide bridged coupling of the blocking reagent. Preferred blocking reagents for that type of blocking reaction are oxidized glutathione (GSSG), cysteine and cystamine. Whereas with cysteine no additional net charge is introduced into the PEGylated protein, the use of the blocking reagents GSSG or cystamine results in an additional negative or positive charge.

The further purification of the compounds of formula (III), including the separation of mono-, di- and tri-PEGylated PEX species, may be done by methods known in the art, e.g. size exclusion, column chromatography.

It was surprisingly found that PEGylated PEX can be purified by ion exchange chromatography in a specifically advantageous manner. The cation exchanger material consists of an SP-Sepharose (Pharmacia, Uppsala, Sweden). Mono-PEGylated PEX was separated from unPEGylated and polyPEGylated PEX in a salt gradient elution (from 20 mM NaCl to 1 M NaCl, 50 mM Tris, pH 8.5).

The percentage of mono-PEG conjugates as well as the ratio of mono- and di-PEG species can be controlled by pooling broader fractions around the elution peak to decrease the percentage of mono-PEG or narrower fractions to increase the percentage of mono-PEG in the composition. About ninety percent mono-PEG conjugates is a good balance of yield and activity. Sometimes compositions in which, for example, at least ninety-two percent or at least ninety-six percent of the conjugates are mono-PEG species (n equals 1) may be desired. In an embodiment of this invention the percentage of conjugates where n is 1 is from ninety percent to ninety-six percent.

Pharmaceutical Formulations

Pegylated PEX can be administered as a mixture, or as the ion exchange chromatography or size exclusion chromatography separated different PEGylated species. The compounds of the present invention can be formulated according to methods for the preparation of pharmaceutical compositions which methods are known to the person skilled in the art. For the production of such compositions, PEGylated PEX according to the invention is combined in a mixture with a pharmaceutically acceptable carrier. Such acceptable carriers are described, for example, in Remington's Pharmaceutical Sciences, 18^{th} edition, 1990, Mack Publishing Company, edited by Oslo et al. (e.g. pp. 1435-1712). Typical compositions contain an effective amount of the substance according to the invention, for example from about 0.1 to 100 mg/ml, together with a suitable amount of a carrier. The compositions may be administered parenterally.

This invention further provides pharmaceutical compositions containing conjugates described herein in which the percentage of conjugates in which n is 1, 2 and/or 3 is preferably at least ninety percent, more preferably at least ninety-two percent.

The pharmaceutical formulations according to the invention can be prepared according to known methods in the art. Usually, solutions of PEGylated PEX are dialyzed against the buffer intended to be used in the pharmaceutical composition and the desired final protein concentration is adjusted by concentration or dilution.

Such pharmaceutical compositions may be used for administration for injection or infusion and contain an effective amount of the monoPEGylated PEX together with pharmaceutically acceptable diluents, preservatives, solubilizers, emulsifiers, adjuvants and/or carriers. Such compositions include diluents of various buffer contents (e.g. arginine, acetate, phosphate), pH and ionic strength, additives such as detergents and solubilizing agents (e.g. Tween™ 80/polysorbate, pluronic™ F68), antioxidants (e.g. ascorbic acid, sodium metabisulfite), preservatives (Timersol™, benzyl alcohol) and bulking substances (e.g. saccharose, mannitol), incorporation of the material into particulate preparations of polymeric compounds such as polylactic acid, polyglycolic acid, etc. or into liposomes. Such compositions may influence the physical state stability rate of release and clearance of the monoPEGylated PEX according to the invention.

Dosages and Drug Concentrations

Typically, in a standard cancer treatment regimen, patients are treated with dosages in the range between 0.1 to 30 mg of PEGylated PEX per kg per day over a certain period of time, lasting from one day to about 30 days or even longer. Drug is applied as a single daily subcutaneous or i.v. bolus injection or infusion of a pharmaceutical formulation containing 0.1 to 10 mg PEGylated PEX per ml. This treatment can be combined with any standard (e.g. chemotherapeutic) treatment, by applying PEGylated PEX before, during or after the standard treatment. This results in an improved outcome compared to standard treatment alone.

The following examples, references and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 The functional elements (e.g. selection marker, origin of replication and expression cassette, promoter, gene to be expressed and transcription terminator)) of the expression plasmid pDS-PEX-2.

FIG. 2 Plasma concentration of 20 kDa PEGylated PEX in mice after 1× s.c. administration of 40 mg/kg.

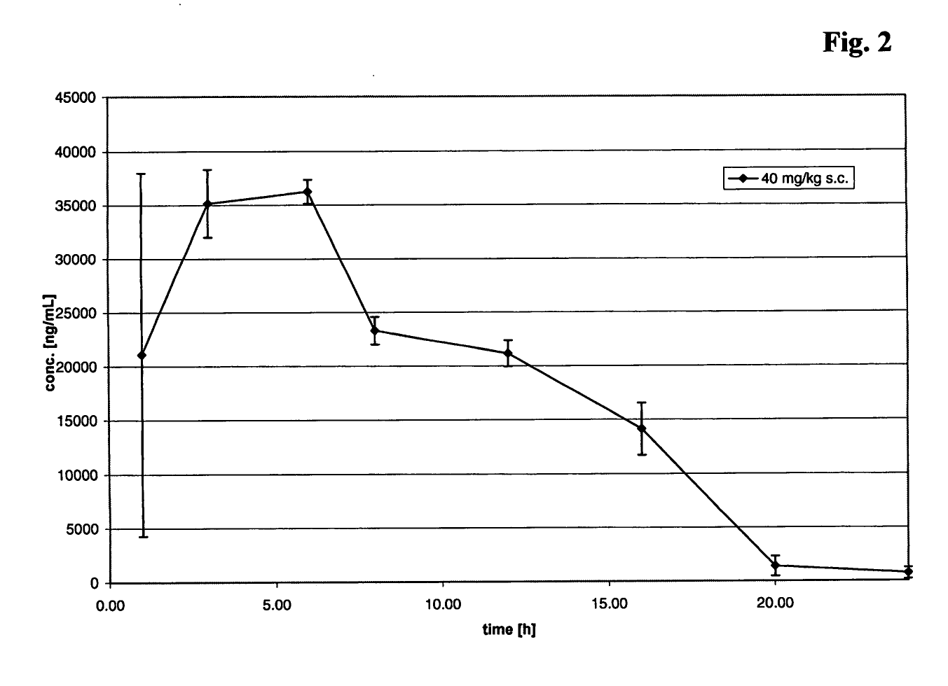

FIG. 3 Comparison of plasma concentration (PEX unmodified, 5 kDa PEGylated PEX, 20 kDa PEGylated PEX) in mice after 1× s.c. administration of 40 mg/kg.

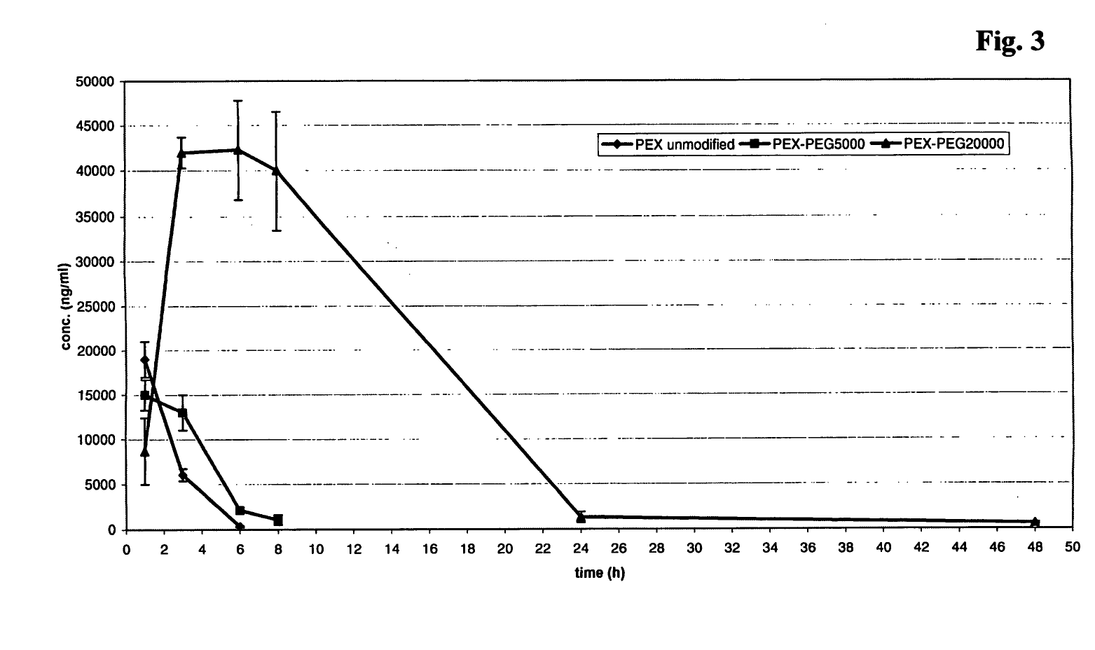

### EXAMPLE 1

### Design and Expression of Recombinant PEX

Material and Methods:

The cDNAs of the 195 amino acid residues comprising C-terminal hemopexin-like domain of MMP-2 PEX (amino acid residues 451-660; SWISS-PROT Accession Number P08253) was isolated by PCR from commercially available human cDNA libraries.

Vector Constructions:

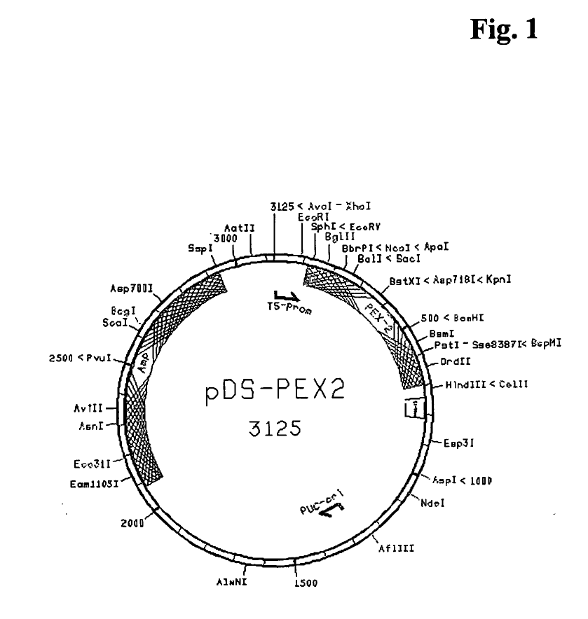

The gene fragments/structural genes to be expressed were amplified by PCR. The gene fragments were provided by singular endonuclease restriction sites suitable for subcloning by means of the 5′-overhanging ends of the PCR primers. If necessary, an ATG translation initiation codon and an “optimized ATG translation initiation region” was introduced by the same technique. The prepared expression plasmids, the base vector and the restriction sites used for cloning are depicted in FIG. 1. The final expression plasmids were identified by restriction mapping and the DNA sequences of the subcloned genes verified by DNA sequencing.

Cell Culture:

E. coli strains were grown in shake flasks in LB or DYT medium supplemented with appropriate antibiotic(s) at temperatures between 20-37° C. The cells were induced with 1-5 mM IPTG for IB formation and 0.1 mM IPTG for soluble protein synthesis, respectively.

IB-Preparation:

IB-supply was accomplished by cell culture in a 10 l fermenter. The PEX protein synthesized in E. coli was homogeneous and found exclusively in the insoluble cell debris fraction (IBs). The expression yield was between 10-50% relative to the total E. coli protein. The estimated expression yield was 2.8 g/l dry weight and a purity of 80% of total protein.

Expression Analysis:

The recombinant expression products were analyzed in E. coli cell lysates by SDS PAGE and staining with Coomassie brilliant blue dye with respect to the
apparent molecular weight,
expression yield, and
solubility (IB-formation).

In general, the cell lysates were processed by centrifugation into a soluble supernatant fraction and an insoluble cell pellet fraction. The latter was resuspended/extracted with 1×SDS sample buffer containing 6-8 M of urea.

### EXAMPLE 2

### Refolding of Recombinant PEX from E. coli

20 g IB preparation is dissolved in 100 ml of solubilization buffer (6 M guanidinium hydrochloride, 2 mM EDTA, 10 mM DTT, 100 mM Tris, pH 7.7) at room temperature and refolded with renaturation buffer (0.6 M arginine, 100 mM Tris, 5 mM CaCl₂, 5 mM GSSG, 1 mM GSH, pH 7.5) at 4° C.

The renaturated protein is dialyzed overnight in a dialysis tube against a buffer of 50 mM Tris, 5 mM CaCl₂, 20 mM NaCl, pH 7.2).

The protein mixtures is purified by means of an SP Sepharose (Pharmacia).

| | |
|---|---|
| Buffer A: | 50 mM Tris, 5 mM CaCl₂, 20 mM NaCl, pH 7.2 |
| Buffer B: | 50 mM Tris, 5 mM CaCl₂, 1 M NaCl, pH 7.2 |
| Gradient: | 0-50% B in 20 CV (column volume) |

The desired fractions are pooled and the concentration is determined at UV 280 nm.

### EXAMPLE 3

### PEGylation of PEX Using PEG-SPA MW 20,000

Present purified PEX is adjusted at a concentration of 0.4 mg/ml using 50 mM Tris, 20 mM NaCl, 5 mM CaCl₂, pH 7.2 (pH range: 7-9).

The protein is mixed with mPEG-SPA (MW 20,000, Shearwater No. 2M4M0P01) at a molecular ratio of 1:1 and incubated for 1 h at room temperature during stirring. The reaction is stopped by addition of ethanol amine (10 μl per 100 ml sample batch) and incubation is again performed for 1 h during stirring.

The sample is dialyzed overnight against 50 mM Tris, 20 mM NaCl, pH 8.5.

### EXAMPLE 4

### Purification

| | |
|---|---|
| Material: | SP-Sepharose (Pharmacia) |
| Buffer A: | 50 mM Tris, 20 mM NaCl, pH 8.5 |
| Buffer B: | 50 mM Tris, 1 M NaCl, pH 8.5 |
| Gradient: | 0-40% B in 80 CV |
| Band detection: | SDS PAGE |
| Determination of concentration: | UV 280 nm |

### EXAMPLE 5

### Pharmacokinetics

Plasma Levels of 20 kDa PEGPEX in Mice after s.c. Administration

| | |
|---|---|
| Compound: | 20 kDa PEGPEX |
| Species: | mouse |
| Gender: | female |
| Strain: | NMRI |
| Dosage: | 40 mg/kg |
| Administered volume: | 10 ml/kg |
| Administration route: | s.c. |
| Feeding status: | fasted overnight, water ad libitum, feeding four |
| | hours after administration |

Blood collection (ca. 0.2 ml per collection time point) at the time points indicated (X). Plasma samples were obtained from blood by centrifugation at room temperature. Plasma samples stored frozen at −20° C. until analysis.

| Time | Group I | | | | Group II | | | | Group III | | | |
|---|---|---|---|---|---|---|---|---|---|---|---|---|
| after | substitute mouse 19 g | | | | substitute mouse 20 g | | | | substitute mouse 19 g | | | |
| administration | M1 | M2 | M3 | M4 | M5 | M6 | M7 | M8 | M9 | M10 | M11 | M12 |
| (h) | 16 g | 19 g | 20 g | 20 g | 19 g | 19 g | 18 g | 19 g | 21 g | 21 g | 17 g | 25 g |
| 1 | X | X | X | X | | | | | | | | |
| 3 | | | | | X | X | X | X | | | | |
| 6 | | | | | | | | | X | X | X | X |
| 8 | X | X | X | X | | | | | | | | |
| 12 | | | | | X | X | X | X | | | | |
| 16 | | | | | | | | | X | X | X | X |
| 20 | X | X | X | X | | | | | | | | |
| 24 | | | | | X | X | X | X | | | | |

Pharmacokinetic Data of 20 kDa PEG-PEX in Female Mice after s.c. Administration:

| | | |
|---|---|---|
| Species: | | mouse |
| Strain: | | NMRI |
| Gender: | | female |
| Number of animals: | | 4 |
| Formulation: | | solution in 0.9% saline |
| ERN: | | — |
| Dose: | mg/kg | 40 |
| Route of administration: | | s.c. |
| CMAX | ng/mL | 36200 |
| CMAX_NORM | ng/mL / mg/kg | 910 |
| TMAX | h | 6 |
| AUC_0_LST | h.ng/mL | 428000 |
| AUC_0_LST_NORM | h.ng/mL / mg/kg | 10800 |
| AUC_0_INF | h.ng/mL | 430000 |
| AUC_0_INF_NORM | h.ng/mL / mg/kg | 10800 |
| PCT_AUC_EXTRA | % AUC extrapolated | 0.518 |
| MRT_LST | h | 8.09 |
| MRT_INF | h | 8.19 |
| CL_TOTAL | mL/min/kg | |
| CL_TOTAL_CTG | L, M, H | |
| CL_ORAL | mL/min/kg | 1.55 |
| VZ | L/kg | |
| VZ_ORAL | (Vz/F) L/kg | 0.296 |
| VSS | L/kg | |
| VSS_CTG | L, M, H | |
| HALFLIFE_Z | h | 2.21 |
| F | % | |

### LIST OF REFERENCES

Bello, L., et al., Cancer Res. 61 (2001) 7501-7506
Brooks, P., et al., Cell 85 (1996) 683-693
Brooks, P., et al., Cell 92 (1998) 391-400
Clark, R., et al., J. Biol. Chem. 271 (1996) 21969-21977
Cockett, M. I., et al., Biochem. Soc. Symp. 63 (1998) 295-313
Collier, I. E., et al., Genomics 9 (1991) 429-434
Cunningham, B. C., et al., Science 254 (1991) 821-825
DE 39 24 705
Docherty, A. J., and Murphy, G., Ann. Rheum. Dis. 49 (1990) 469-479
EP-A 0 473 084
Goodson, R. J., and Katre, N. V., BioTechnology 8 (1990) 343-346
Grasetti, D. R,. and Murray, J. F. in J. Appl. Biochem. Biotechnol. 119 (1967) 41-49
Hermanson, G. T., in Bioconjugate Techniques, Academic Press, San Diego (1996) p. 147-148
Hershfield, M. S., et al., New England Journal of Medicine 316 (1987) 589-596
Katre, N. V., J. Immunol. 144 (1990) 209-213
March, J., Advanced Organic Chemistry (1977) 375-376
Meyers, F. J., et al., Clin. Pharmacol. Ther. 49 (1991) 307-313
Monfardini, C., et al., Bioconjugate Chem. 6 (1995) 62-69
Morpurgo, M., et al. J. Bioconj. Chem. 7 (1996) 363 ff
Murphy, G., et al., APMIS 107 (1999) 33-44
Nagase, H., and Woessner, J. F., J. Biol. Chem. 274 (1999) 21491-21494
Nicolson, G. L., et al., Curr. Opin. Cell Biol. 1 (1989) 1009-10019
Pfeifer, A., et al., Proc. Natl. Acad. Sci. USA 97 (2000) 12227-12232
Remington's Pharmaceutical Sciences, 18th edition, 1990, Mack Publishing Company, edited by Oslo et al. (e.g. pp. 1435-1712)
Silletti, S., et al., Proc. Natl. Acad. Sci. USA 98 (2001) 119-124
Stetler-Stevenson, W. G., et al., Annu. Rev. Cell Biol. 9 (1993) 541-573
Swiss-Prot Database No. P08253
Tanaka, H., et al., Cancer Research 51 (1991) 3710-3714
U.S. Pat. No. 5,382,657
U.S. Pat. No. 5,672,662
U.S. Pat. No. 5,932,462
Veronese, F. M., Biomaterials 22 (2001) 405-417
WO 94/01451
Woessner, J. F., Jr., FASEB J. 5 (1991) 2145-2154
Wojchowski, D. M., et al., Biochim. Biophys. Acta 910 (1987) 224-232

1. A conjugate comprising the C domain of human gelatinase A (PEX domain, PEX) and one to three poly(ethylene glycol) group (s), said poly(ethylene glycol) group (s) having an overall weight of from about 10 to 40 kDa and being conjugated via primary amino group (s) of said PEX.

2. (canceled)

3. (canceled)

4. (canceled)

5. (canceled)

6. (canceled)

7. (canceled)

8. (canceled)

9. (canceled)

10. (canceled)

11. (canceled)

12. The conjugate of claim 1 wherein said poly(ethylene glycol) group (s) is/are monomethoxy poly(ethylene glycol) group (s).

13. The conjugate of claim 1 wherein it contains one poly(ethylene glycol) group.

14. The conjugate of claim 1 wherein the poly(ethylene glycol) group (s) is/are branched poly(ethylene glycol) group(s).

15. The conjugate of claim 1 wherein said conjugate is of the formula
P—[NHCO—(CH)ₓ—(OCH₂CH₂)ₘ—OR]ₙ I
and wherein
x is 2 or 3;
m is from about 220 to about 900 dependent on the molecular weight of the poly(ethylene glycol) group(s);
n is 1 to 3;
P is said PEX without the n amino group(s) which form amide linkage(s) with the poly(ethylene glycol) group(s).

16. The conjugate of claim 1 wherein said conjugate is of the formula
P—[NH—CO—X—S—Y—(OCH₂CH₂)ₘ—OR]ₙ I
and wherein
m is from about 220 to about 900 dependent on the molecular weight of the poly(ethylene glycol) group(s);
n is 1 to 3;
R is a linear or branched alkyl residue having one to six carbon atoms;
X is —(CH₂)ₖ— or —CH₂(O—CH₂—CH₂)ₖ— wherein k is from 1 to about 10;
Y is selected from 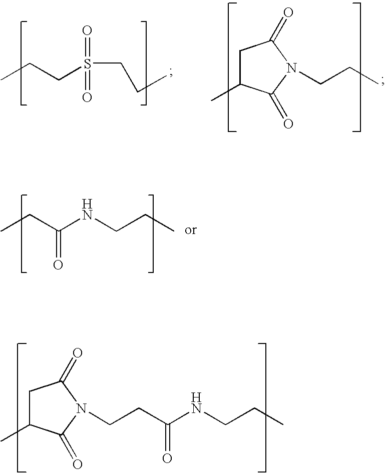
and P is PEX without the n amino group(s) which form amide linkage(s) with the poly(ethylene glycol) group(s).

17. A method for the preparation of a conjugate comprising the C domain of human gelatinase A (PEX domain, PEX) and one to three poly(ethylene glycol) group(s) having an overall molecular weight of from 10 to 40 kDa and being conjugated via primary amino group(s) of said PEX, said method comprising the reaction of said PEX with said polyethylene glycol under conditions whereby one to three polyethylene glycol groups are chemically bound to the primary amino group(s) of PEX.

18. A pharmaceutical composition comprising a conjugate of the C domain of human gelatinase A (PEX domain, PEX) and one to three poly(ethylene glycol) group (s), said poly(ethylene glycol) group (s) having an overall weight of from about 10 to 40 kDa and being conjugated via primary amino group (s) of said PEX together with a pharmaceutically acceptable carrier.

19. A method for treating cancer which comprises administering to a patient in need thereof, a therapeutically effective amount of a conjugate comprising the C domain of human gelatinase A (PEX domain, PEX) and one to three poly(ethylene glycol) group (s), said poly(ethylene glycol) group (s) having an overall weight of from about 10 to 40 kDa and being conjugated via primary amino group (s) of said PEX.
